(19) United States
(12) Patent Application Publication
(10) Pub. No.: US 2022/0275931 A1
(43) Pub. Date: Sep. 1, 2022

(54) AIR TERMINAL DEVICE WITH INTEGRAL LIGHT FIXTURE

(71) Applicant: HALTON OY, Kausala (FI)

(72) Inventor: Kim Hagström, Kausala (FI)

(73) Assignee: HALTON OY, Kausala (FI)

(21) Appl. No.: 17/682,618

(22) Filed: Feb. 28, 2022

(30) Foreign Application Priority Data
Mar. 1, 2021 (EP) 21159866.9

(51) Int. Cl. F21V 33/00; F21V 17/10; A61L 9/20

(52) U.S. Cl. CPC F21V 33/0088; F21V 17/107; A61L 9/20; A61L 2209/12

(57) ABSTRACT

An air terminal device with integral light fixture for a clean room, comprising a housing having a cover panel comprising at least one opening, a light fixture for providing light outside of the air terminal device, an air chamber configured to receive air and to discharge air out of the air chamber, wherein the air is configured to be transferred through the at least one opening in the cover panel, wherein the light fixture is arranged inside the air chamber, and the cover panel comprises at least one light fixture opening, in which the light fixture is integrated.

### TECHNICAL FIELD

The present invention relates to an air terminal device with integral light fixture for a clean room.

### BACKGROUND OF THE ART

A clean room, such as a room for research or production or room in a hospital, such as operating room or patient room, has high hygiene demands. Such rooms have need for both air purification and surface cleaning. In traditional approach air purification and surface cleaning by irradiation (UV, Blue light) as well as room lighting are used with separate systems with individual installation. In above mentioned cleanrooms constructional tightness—especially ceiling tightness and product maintainability are essential requirements to ensure clean operations. Having separate systems add significant complexity and challenge to ceiling design and integration. Firstly, a hole in the ceiling is a source for leakage and impairs the loadbearing capability of the ceiling. Secondly, the air supply and exhaust arrangements are the top priority in the rooms with highest cleanliness, and any other ceiling installation add challenge to room airflow control.

Typically, the disinfection and lighting arrangements are used continuously at a high intensity. The light fixtures are installed in a separate enclosure next to an air device and they are continuously at a high temperature, which pose a risk for shortened lifetime for light fixtures. Also, in such an arrangement light fixture maintenance would require access to ceiling void by opening the ceiling construction.

### OBJECTIVE OF THE INVENTION

The objective of the device is to alleviate the disadvantages mentioned above.

In particular, it is an objective of the present device to provide an air terminal device with efficient air diffusion and surface disinfection capabilities.

### SUMMARY

According to a first aspect, the present invention provides an air terminal device with integral light fixture for a clean room, comprising a housing having a cover panel comprising at least one opening, a light fixture for providing light outside of the air terminal device, an air chamber configured to receive air and to discharge air out of the air chamber. The air is configured to be transferred through the at least one opening in the cover panel. The light fixture is arranged inside the air chamber, and the cover panel comprises at least one light fixture opening, in which the light fixture is integrated.

The advantage of the invention is that air diffusion and surface disinfection may be provided efficiently with one integrated device.

In an embodiment of the device, the light fixture is arranged within the light fixture opening so that the perimeter of the light fixture is at the same level as the cover panel or projecting from the cover panel. The advantage is that the light may be distributed on wider area and even along the surface of the cover panel. Thus, the light may be provided as with using separate lighting.

In an embodiment of the device, the cover panel is detachably connected to the housing whereby the air chamber and the light fixture is exposed when the cover panel is removed. The advantage is that the air chamber and the light fixture are accessible without need to disassemble the device or access to the contaminated ceiling void.

In an embodiment of the device, the cover panel is hingedly connected to the housing.

In an embodiment of the device, the light fixture is detachable. The advantage is that the light fixture may be replaced and the space behind the light fixture is accessible without need to access the contaminated ceiling void.

In an embodiment of the device, the light fixture is hingedly connected to the air terminal device.

In an embodiment of the device, the light fixture is configured to be flushed with the received air before the air is diffused out of the air terminal device. The advantage is that the lifetime of the light fixture may be extended when the heat from the light fixture is removed by the air flow. Especially when cooled supply air is transferred through the air terminal device, the cooled supply air removed heat from the light fixture efficiently.

In an embodiment of the device, the cover panel comprises plurality of openings configured to provide adjustable air flow pattern. Advantage is that the air flow pattern may be adjusted to meet the requirements of the clean room.

In an embodiment of the device, the device comprises a filter configured to filter the air received into the air chamber. The advantage is that the impurities may be removed from the air before it is discharged into the clean room.

In an embodiment of the device, the filter is inside the air chamber. The advantage is that the filter is more easily accessible.

In an embodiment of the device, the light fixture is between the filter and the cover panel. The advantage is that the air is filtered before the light fixture is flushed with the air flow and, therefore, the light fixture remains cleaner and the impurities are not collected on the surface of the light fixture, which would increase the temperature of the light fixture even further.

In an embodiment of the device, the light fixture is configured to provide light to the filter. The advantage is that the filter may be disinfected by the same light fixture and, thus, the filter remains cleaner and/or the filter needs to be replaced less frequently.

In an embodiment of the device, the light fixture is configured to produce visible light.

In an embodiment of the device, the light fixture is configured to produce disinfection light.

In an embodiment of the device, the light fixture is configured to produce visible light and disinfection light.

In an embodiment of the device, the device comprises a control unit configured to adjust the light fixture to produce visible light or disinfection light.

In an embodiment of the device, the air chamber is a supply air chamber configured to receive supply air and to discharge the supply air out of the device through the at least one opening of the cover panel.

It is to be understood that the aspects and embodiments of the invention described above may be used in any combination with each other. Several of the aspects and embodiments may be combined together to form a further embodiment of the invention.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 shows an air terminal device according to the invention,

FIG. 2 shows the air terminal device wherein the cover panel is removed completely,

FIG. 3 shows the air terminal device wherein the cover panel is removed, and the light fixture is pivoted, with a hinge,

FIG. 4 is a cross-sectional view of the air terminal device from one end.

FIG. 5 is a cross-sectional view of the air terminal device from one end, wherein the device comprises a filter,

FIG. 6 shows an embodiment of the air terminal device, wherein the device comprises two cover panels and two light fixtures, and

FIG. 7 shows an embodiment of the air terminal device, wherein the device comprises several cover panels and several light fixtures, and the device is configured in rectangular form.

### DETAILED DESCRIPTION

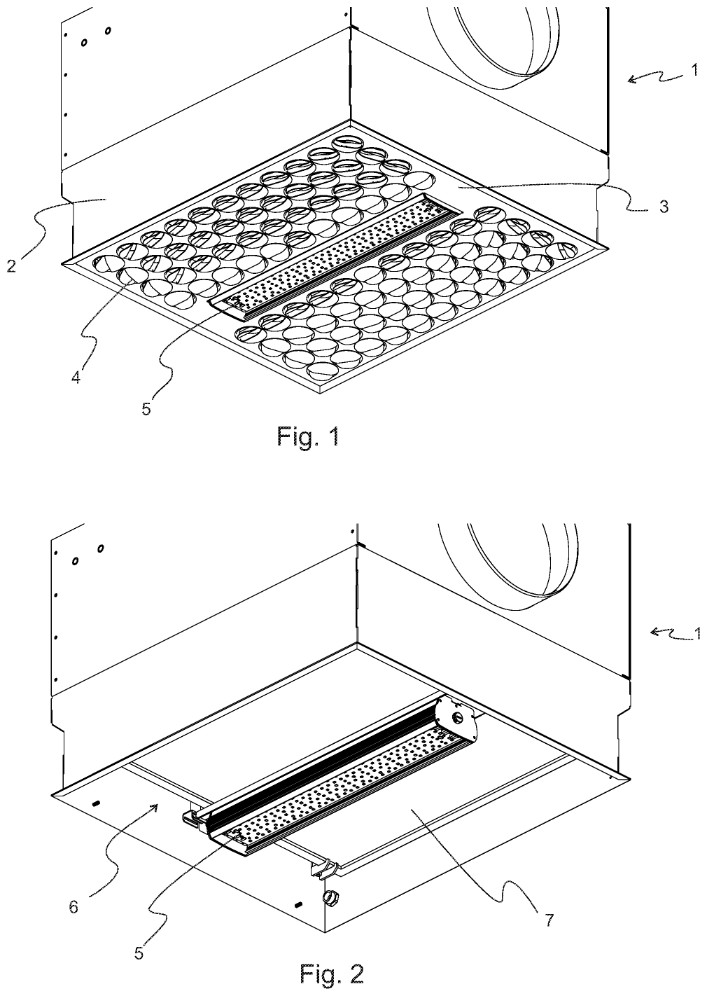

FIG. 1 shows an air terminal device 1 with integral light fixture 5 for a clean room. The air terminal device may be a supply air device for providing supply air into the clean room or it may be exhaust air device for removing room exhaust air from the clean room. In both applications, the basic structure of the device is similar, and the air flow direction is the only one that changes. For air supply, the air is received from, for example, a supply air duct and discharged into the clean room, and for air exhaust, the exhaust air is received from the clean room and discharged into, for example, an exhaust duct.

The air terminal device comprises a housing 2 which forms the outer casing of the device. The housing 2 comprises a cover panel 3, which is exposed to the clean room when the device is installed in the clean room. The cover panel 3 comprises at least one opening 4, through which air is transferred into the air terminal device or out of the air terminal device. The air transferred through the at least one opening 4 may be exhaust room air from the clean room or the air may be supply air from supply air duct of the building. The cover panel 3 may comprise plurality of openings 4, as seen in FIG. 1. The air terminal device comprises an air chamber 6 which is arranged to receive air and to discharge the air out of the air chamber 6. The air chamber 6 is covered by the cover panel 3 and the cover panel 3 is detachably connected to the housing 2, i.e. the cover panel 3 is openable. The cover panel 3 may be connected to the housing 2 so that it may be completely removed from the air terminal device or it may be connected to the housing 2 by hinge or hinges so that it may be removed by pivoting it from one edge while one edge remains connected to the housing 2 by the hinge or hinges. In any case, removing (completely or hingedly) the cover panel 3 exposes the air chamber 6 of the air terminal device.

The cover panel 3 may be perforated panel having plurality of perforations, through which the supply air may be discharged into the room or room the exhaust air from the room may be received into the air terminal device.

In case that the air terminal device is for providing a supply air into the clean room, the openings 4 in the cover panel 3 may be used for providing desired air flow pattern from the device. The openings 4 of the cover panel 3 may comprise rotatable nozzles for directing the air jets. The clean rooms are used for highly critical operations and, therefore, it is beneficial to have possibility to adjust the air flow pattern so that air flows are directed in desired directions.

The air terminal device may comprise a plenum 10 which is configured to provide supply air to the air chamber 6 or to receive room exhaust air from the air chamber 6. Optionally, the plenum 10 may be configured to provide air to several air chambers 6 in case that the air terminal device comprises one plenum 10 and several air chambers 6, which embodiments will be discussed later.

The air terminal device comprises a light fixture 5 for providing light outside of the air terminal device, i.e. into the clean room, and the light fixture 5 is arranged inside the air chamber 6 and integrated with a light fixture 5 opening. This should be understood so that the light fixture 5 is mainly inside the air chamber 6 and some parts, e.g. light diffuser or cover, may be at the light fixture opening or even projecting from the cover panel 3, if such components are part of the light fixture 5. The main parts, e.g. light source and electricity are inside the air chamber 6. The cover panel 3 comprises at least one light fixture opening in which the light fixture 5 is integrated, and the light is provided outside of the air terminal device i.e. into the clean room. The light fixture 5 may be integrated to the cover panel 3 so that the light fixture 5 may be removed from the device by removing the cover panel 3. Optionally, the light fixture 5 is connected to the housing and by removing the cover panel 3, the light fixture 5 inside the air chamber 6 is completely exposed and the light fixture 5 may be removed from the air terminal device or it may be inspected or the lightning source, e.g. lamp, may be replaced. By placing the light fixture 5 inside the air chamber 6, the air flow inside the air chamber 6 may be used for removing heat, i.e. cooling, from the light fixture 5, i.e. the light fixture 5 is flushed by the air, and, thus, preventing overheating of the light fixture 5. As the light fixture 5 may be kept at lower temperature, the lifetime of the light fixture 5 may be extended. When the air terminal device is a supply air device, the supply air may be cooled air, which is transferred through the device. The cooled supply air removes heat efficiently from the light fixture when the light fixture is flushed with the cooled supply air.

The light fixture 5 comprises a light source or light sources, which may comprise a light-emitting diode (LED) unit having a number of LEDs. The light fixture 5 may be configured to provide visible light (wavelengths in range of 400-700 nm) or disinfection light, such as blue light (wavelengths in range of 400-490 nm) or UV light (wavelengths in range of 220-300 nm), which have been shown to destroy impurities, such as bacteria. Optionally, the light fixture 5 may be configured to provide visible light and disinfection light. The light fixture 5 may be configured to provide disinfection light at least on two different wavelengths.

The air terminal device 1 may comprise a control unit, which may be configured adjust the operation of the air terminal device 1. The control unit may be configured to adjust the light fixture 5 to produce visible light or disinfection light based on the schedule of the clean room. For example, the visible light and the disinfection light may be configured to be produced based on schedule of operations, i.e. disinfection light is produced when disinfection is needed and visible light when only visible light is needed. The control unit may also be configured to adjust and/or schedule the air ventilation together with the light fixture, i.e. supplying the supply air into the room or exhaust room air into the air terminal device.

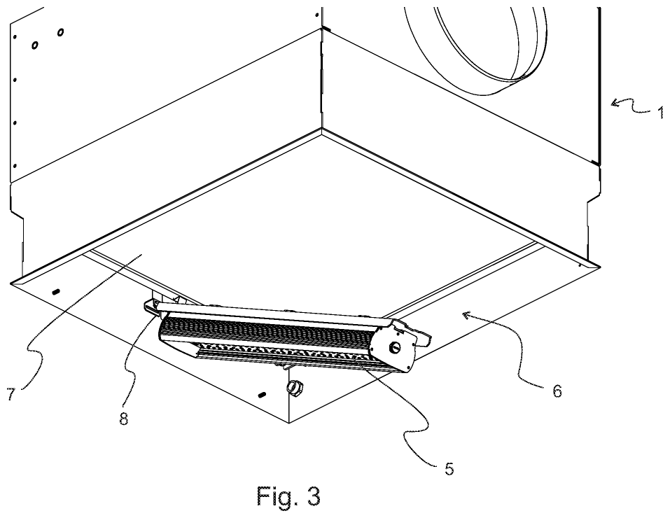

FIG. 2 shows the air terminal device 1 wherein the cover panel 3 is removed completely, and the air chamber 6 and the light fixture 5 is exposed. The light fixture 5 may be detachable so that it may be removed completely and/or replaced if needed. The light fixture 5 may be connected to the frame, e.g. to opposite side walls of the air chamber 6, by hinge 8 or hinges so that it may be pivoted from one end while the other end remains connected to the frame by the hinge 8 or hinges.

The air terminal device 1 may comprise a filter 7, which is configured to filter the air received into the air chamber 6 before it is discharged into the clean room. The filter 7 may be inside the air chamber 6 or it may be outside of the air chamber 6 so that the air is filtered before it is received into the air chamber 6. By filtering the air, impurities, such as dust, particles, bacteria, viruses etc. may be removed from the air and the discharged air meets the requirements regarding cleanliness or the supply air.

The filter 7 may be placed in parallel to the cover panel 3 so that it covers the whole cross-sectional area of the air chamber 6. Thus, the filter 7 surface area may be larger and, thus, its capability to filter impurities is better.

The filter 7 may be high-efficiency particulate air (HEPA) filter.

When the filter 7 is placed inside the air chamber, the light fixture may be configured to provide light to the filter 7 for disinfecting the filter 7. Optionally, another separate light fixture may be provided to provide disinfection light to the filter 7.

FIG. 3 shows the air terminal device 1 wherein the cover panel 3 is removed, and the light fixture 5 is pivoted from one end. By removing the cover panel 3 and the light fixture 5, the filter 7 is accessible, and it may be removed, inspected, or replaced without disassembling the whole air terminal device 1 or without need to access inside the ceiling.

The light fixture 5 may be arranged between the filter 7 and the cover panel 3, whereby the impurities in the air are filtered before the light fixture 5 and the light fixture 5 remains cleaner.

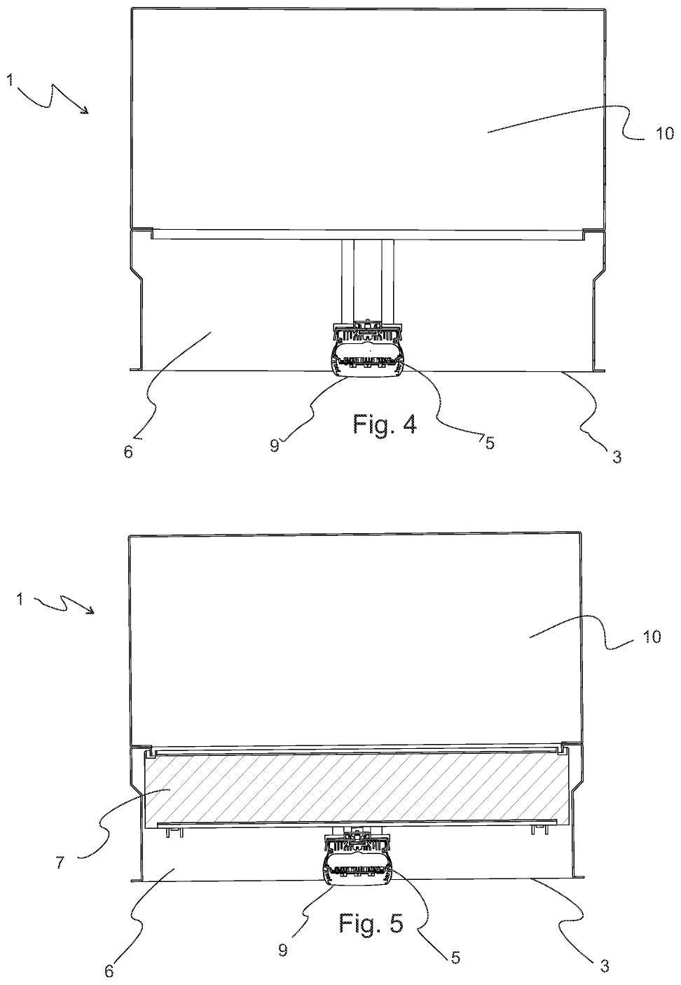

FIG. 4 is a cross-sectional view of the air terminal device 1 viewed from one end. The light fixture 5 is arranged inside the air chamber 6 so that the air flow inside the air chamber 6 flushes and, thus, removes heat from the light fixture 5. The light fixture 5 may be arranged so that the perimeter 9 of the light fixture 5 is at the same level as the cover panel 3, or it may be arranged so that the perimeter 9 of the light fixture 5 projecting from the cover panel 3, as seen in FIG. 4. If the perimeter 9 of the light fixture 5 is projecting from the cover panel 3, the light may be provided to larger area and even along the cover panel 3 surface.

FIG. 5 is the same cross-sectional view of the air terminal device 1 as in FIG. 4. In this figure, the filter 7 is provided inside the air chamber 6 and the light fixture 5 is between the filter 7 and the cover panel 3.

The features and embodiments discussed above may be implemented in embodiments wherein the air terminal device 1 comprises two or more cover panel 3s and two or more light fixtures 5, and wherein the air terminal device 1 has different shapes, as will be discussed in the following section.

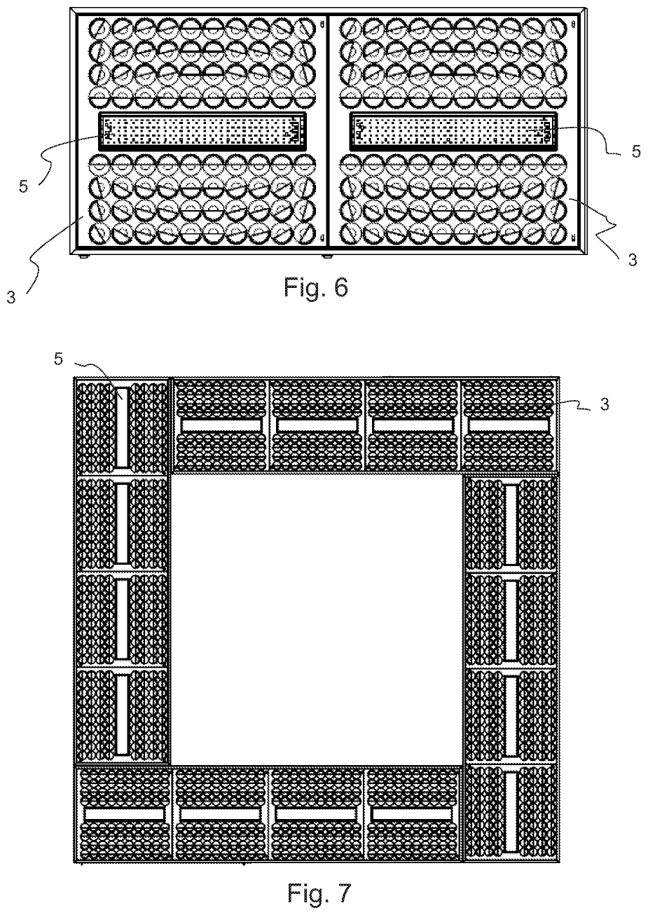

FIG. 6 shows an embodiment of the air terminal device 1. In this embodiment, the air terminal device 1 comprises two cover panels 3 and two light fixtures 5 adjacent to each other. The air terminal device 1 may also comprise more than two cover panels 3 and more than two light fixtures 5. The number of separate cover panels 3 may be the same as the number of separate light fixtures 5. Optionally, each cover panel 3 comprises several light fixture openings through which the light is provided from several light fixtures 5.

The air terminal device 1 having two or more cover panels 3 and two or more light fixtures 5, may comprise only one air chamber 6 and each light fixture 5 is arranged inside this one air chamber 6. Optionally, the air terminal device 1 may comprise several air chambers 6 so that at least one light fixture 5 is arranged inside each air chambers 6.

FIG. 7 shows an embodiment of the air terminal device 1. In this embodiment, the air terminal device 1 comprises several cover panels 3 and several light fixtures 5. The air terminal device 1 is configured in rectangular shape so that it may be installed to the ceiling of a clean room so that it surrounds a critical area, for example operating table in hospital, below. Optionally, the air terminal device 1 may be configured so that it has two or more separate elongated sections, as seen for example in FIG. 6, and they may be installed to the ceiling of a clean room so that a critical area below the air terminal device 1 is between the separate sections, e.g. two sections are on opposite sides of the critical area.

Although the invention has been the described in conjunction with a certain type of device, it should be understood that the invention is not limited to any certain type of device. While the present inventions have been described in connection with a number of exemplary embodiments, and implementations, the present inventions are not so limited, but rather cover various modifications, and equivalent arrangements, which fall within the purview of prospective claims.

1. An air terminal device with integral light fixture for a clean room, comprising:
a housing having a cover panel comprising at least one opening,
a light fixture for providing light outside of the air terminal device,
an air chamber configured to receive air and to discharge air out of the air chamber,
wherein the air is configured to be transferred through the at least one opening in the cover panel, wherein
the light fixture is arranged inside the air chamber, and
the cover panel comprises at least one light fixture opening, in which the light fixture is integrated.

2. The air terminal device according to claim 1, wherein the light fixture is arranged within the light fixture opening so that the perimeter of the light fixture is at the same level as the cover panel or projecting from the cover panel.

3. The air terminal device according to claim 1, wherein the cover panel is detachably connected to the housing whereby the air chamber and the light fixture is exposed when the cover panel is removed.

4. The air terminal device according to claim 1, wherein the cover panel is hingedly connected to the housing.

5. The air terminal device according to claim 1, wherein the light fixture is detachable.

6. The air terminal device according to claim 1, wherein the light fixture is hingedly connected to the air terminal device.

7. The air terminal device according to claim 1, wherein the light fixture is configured to be flushed with the received air before the air is diffused out of the air terminal device.

8. The air terminal device according to claim 1, wherein the cover panel comprises plurality of openings configured to provide adjustable air flow pattern.

9. The air terminal device according to claim 1, comprising a filter configured to filter the air received into the air chamber.

10. The air terminal device according to claim 9, wherein the filter is inside the air chamber.

11. The air terminal device according to claim 9, wherein the light fixture is between the filter and the cover panel.

12. The air terminal device according to claim 10, wherein the light fixture is configured to provide light to the filter.

13. The air terminal device according to claim 1, wherein the light fixture is configured to produce visible light.

14. The air terminal device according to claim 1, wherein the light fixture is configured to produce disinfection light.

15. The air terminal device according to claim 1, wherein the light fixture is configured to produce visible light and disinfection light.

16. The air terminal device according to claim 15, comprising a control unit configured to adjust the light fixture to produce visible light or disinfection light.

17. The air terminal device according to claim 1, wherein the air chamber is a supply air chamber configured to receive supply air and to diffuse supply air out of the device through the at least one opening of the cover panel.
